(19) United States
(12) Patent Application Publication
(10) Pub. No.: US 2021/0145327 A1
(43) Pub. Date: May 20, 2021

(54) Electrochemical Sensors Deployed in Catheters for Subcutaneous and Intraperitoneal Sensing of Glucose and Other Analytes

(71) Applicant: Biorasis, Inc, Storrs, CT (US)

(72) Inventors: Faquir Jain, Storrs, CT (US); Fotios Papadimitrakopoulos, West Hartford, CT (US); Michail Kastellorizios, Fort Worth, TX (US); Allen Legassey, Mansfield, CT (US); Pik-Yiu Chan, Ashford, CT (US)

(21) Appl. No.: 16/411,678

(22) Filed: May 14, 2019

Related U.S. Application Data
(60) Provisional application No. 62/671,407, filed on May 14, 2018.

(51) Int. Cl. A61B 5/145; A61B 5/00; A61M 5/172; A61M 5/142

(52) U.S. Cl. CPC A61B 5/14532; A61B 5/14539; A61B 5/14542; A61M 2205/50; A61B 5/6866; A61M 5/1723; A61M 5/142; A61B 5/14503

(57) ABSTRACT

Biosensing platform deployed in a catheter is described which permits long-term operation of biosensors monitoring glucose and other analytes subcutaneously or intraperitoneally (IP) to manage diabetes. A method for integrating a plurality of biosensors monitoring glucose and other analytes into a catheter platform. The catheter platform comprises of electrochemical sensors, sensor electronics, RF and optical communication devices, as well as pump control electronics to facilitate glucose management. Catheter mounted biosensor is shown with micro-dialysis provisions.

### RELATED APPLICATIONS

This application is related to and claims the benefit of priority of the filing date of U.S. Provisional Patent Application Ser. No. 62/671,407 filed May 14, 2018, the contents of which are incorporated by reference herein in its entirety.

### FIELD OF THE INVENTION

This invention relates generally to an integrated, implantable, biosensor platform deployed in catheters, which permits long-term operation of biosensors monitoring glucose and other analytes subcutaneously and intraperitoneally (IP) to manage diabetes. In addition, methods are described to monitor glucose and other analytes while interfacing with insulin dispensing pumps. The method includes electrochemical sensors, sensor electronics and optical devices, their powering using optical power located in an external unit, RF and/or optical communication devices transmitting analyte levels to an external unit, as well as pump control electronics to facilitate glucose management.

### BACKGROUND OF THE INVENTION

Diabetes is the third leading cause of mortality in the United States^{[1]}. Type 1 diabetes management requires a constant effort from the patients to self-dose insulin multiple times per day. This creates a burden not only on the patient's daily life but also on their health as accurate insulin dosing depends on a multiplicity of factors such as caloric intake, energy levels, time of day, etc. Diabetes-related complications such as cardiovascular disease and amputations may be prevented with tight blood glucose control under strict insulin dose planning regimes. Artificial pancreas systems offer the next generation of diabetes management with the first hybrid system being approved by the FDA in 2016. While this system takes a semi-automatic approach, future generations of artificial pancreas are expected to incorporate greater automation and lesser patient involvement before transitioning to the ideal fully automated systems.

Biosensing platforms, or biosensors, for medical applications have significant promises as a means to diagnose and to manage diabetes and other diseases. A biosensor can be any device that detects any chemical or physical change, converts that signal into an electrical or chemical signal and transmits the response to a secondary device via RF, ultrasound, optical methods. This invention relates to sensing of glucose and other analytes levels subcutaneously and intraperitoneally (IP) to manage diabetes. In the case of subcutaneous layer detection, the sensing elements and a custom integrated circuit can be implemented on an implantable platform which is equipped with wireless powering and communication. In another version, it can be embedded in a catheter for external measurements. The catheter version can be adapted for sensing intraperitoneally (IP).

The design of custom electronic circuits, optical and other devices varies with characteristics specific to either a subcutaneous or an IP-implanted glucose sensing application. For example, in IP space there is constant movement, the opportunity for fast glucose kinetics, and the integration with the fully implantable insulin pump. In the case of subcutaneous sensing using catheter for insulin dispensing (insulin pump location) may either be local in the vicinity of embedded sensors or at a different location of the body separated from the implantable sensor.

### SUMMARY OF THE INVENTION

A biosensing platform deployed in a catheter is described which permits long-term operation of biosensors monitoring glucose and other analytes subcutaneously and intraperitoneally (IP) to manage diabetes.

A method of integrating a plurality of biosensors monitoring glucose and other analytes subcutaneously using ring electrodes forming the electrochemical sensor and integrated with electronic and optical devices on same platform that is implanted subcutaneously. The biosensor platform in one embodiment is housed in a catheter with insulin dispensing capability. For subcutaneous applications, the method integrates coated electrodes with electronic, optical and other components used in powering and communications in an enclosure that protects against body fluids.

Another method describes use of ring electrodes mounted into a catheter platform for intraperitoneally usage. The catheter platform for intraperitoneal usage comprises of electrochemical sensors, sensor electronics, RF and optical communication devices, as well as pump control electronics to facilitate glucose and insulin management.

Catheter based biosensing is also described in this invention using micro-dialysis.

### BRIEF DESCRIPTION OF THE FIGURES

The foregoing and other features and advantages of the present invention should be more fully understood from the accompanying detailed description of illustrative embodiments taken in conjunction with the following Figures in which like elements are numbered alike in the several Figures:

FIG. 1(a). Schematic of an integration method combining coated electrodes with electronic, optical and other components used in powering and communications in an enclosure that protects against body fluids.

FIG. 1(b) Implantable platform where ring electrodes are assembled along with electronic and optical components.

FIG. 1c Si to silicon seal using gold perimeter fence.

FIG. 1(d) shows a ring which is made of Si, Gold or Pt.

FIG. 1(e) shows SOS cover and Si substrate bonding together in a hermetic seal.

FIG. 1(f) shows a compact biosensor platform integrating electrodes on the hermetic package.

FIG. 1(g) schematically shows a biosensor platform integrating electrodes protruding as wires that are coated appropriately to serve as reference, working and counter electrodes.

FIG. 2(a) Schematic representation (not to scale) of a six-electrode glucose sensor.

FIG. 2b. Cross section at one of six electrodes; Components from the center outward are Insulin dispensing catheter (shown White); Silicone multi-lumen tubing (Blue); Platinum wires (inside silicone tubing) and rings wrapped around the silicone tubing (Grey); Glucose sensing layers (Orange); Drug eluting coating embedded on an outer silicone catheter (purple, Green/Purple); And Glucose exchange between the IP fluid and the sensing elements (Red arrows).

FIG. 2(c) Right: 3-Dimensional schematic.

FIG. 3(a) Circuit schematic of electronics interfacing with sensor platform, sensor electronics (MOSIS chip) and insulin pump controller for IP applications.

FIG. 3(b) Photograph of a printed circuit board.

FIG. 4 shows in vitro test set up.

FIG. 5 DATA showing sensor output as a function of glucose concentration.

FIG. 6 Prior ART implantable biosensor.

FIG. 7 Overview of a microdialysis catheter setup for subcutaneous bio-sensing.

FIG. 8(a) Schematic of microdialysis setup which is housed in a catheter. It shows fluid in and fluid out tubes interfacing with permeable microdialysis probe.

FIG. 8(b) Schematic of microdialysis setup housed in a catheter with sensing electrodes and electronic interface.

FIG. 9. External watch-like proximity communicator unit interfacing with subcutaneous implantable sensor and its electronic and optical devices along with insulin dispensing tube housed in a catheter.

### DETAILED DESCRIPTION OF THE FIGURES

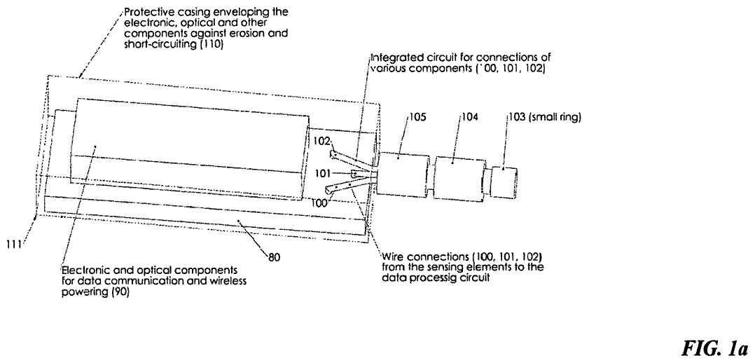

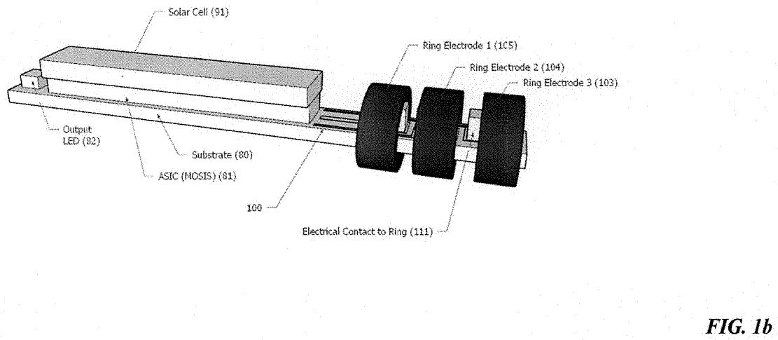

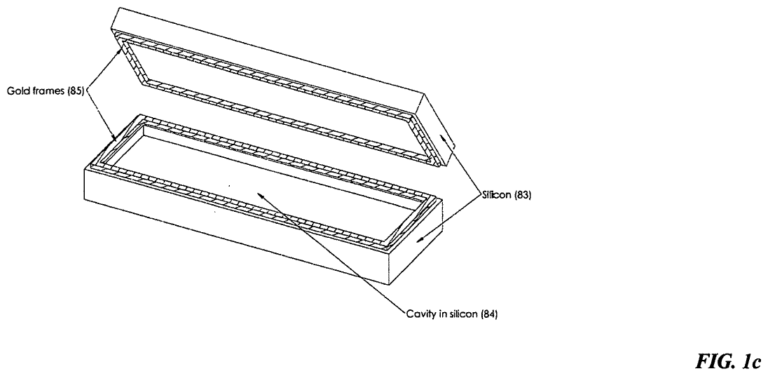

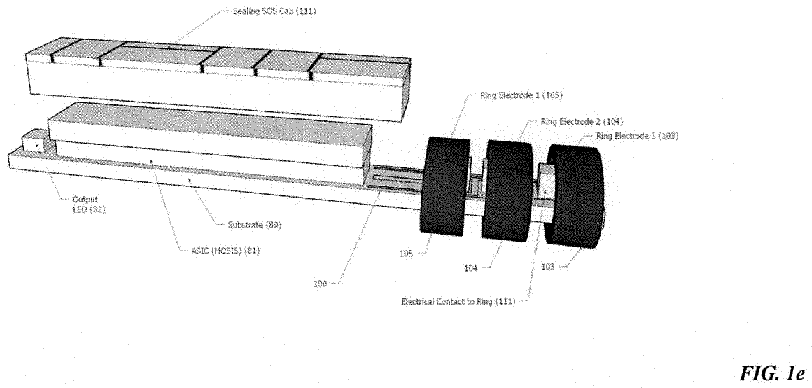

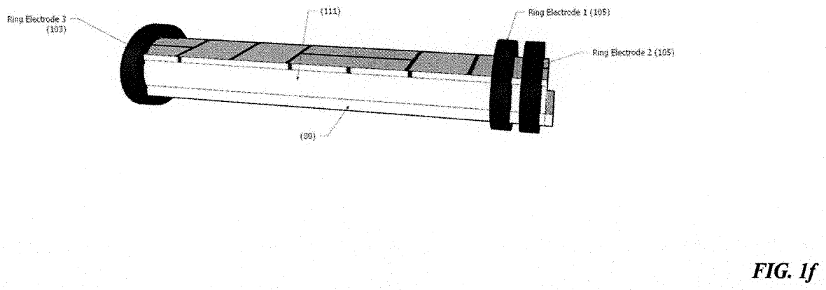

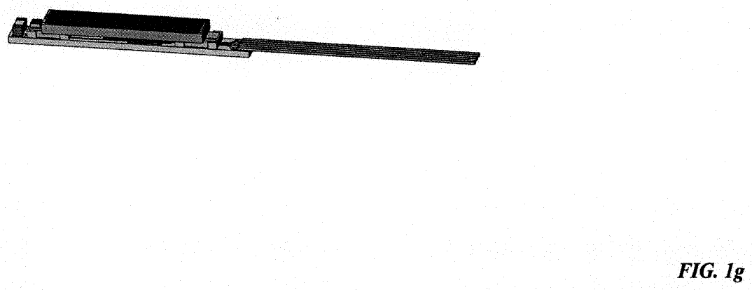

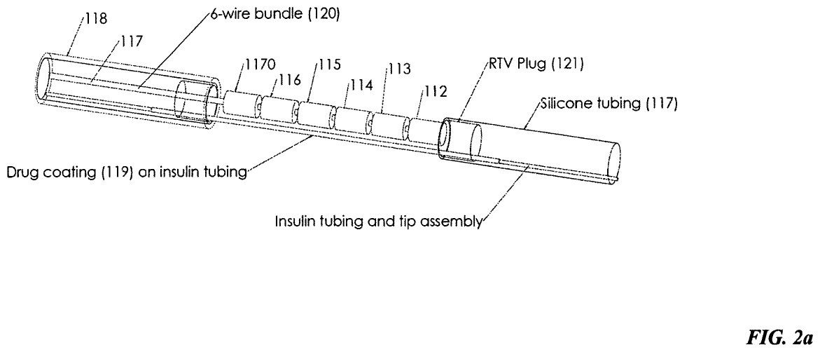

FIG. 1(a). Schematic of an integration method of sensor electrodes combining coated ring electrodes (103, 104, 105) with electronic, optical and other components (90) used in powering and communications in an enclosure (110) that protects against body fluids. Here a heat-shrink tubing (111) is used to protect further the electronic and optical devices. The electrodes are connected electrically using wire connections (100, 101, and 102). The biocompatible coatings are not shown.

FIG. 1(b) An embodiment of biosensor integration schematically shown combining coated ring electrodes (103, 104, 105) with electronic chip (81), optical components used in powering and communications (82 LED and 91 Solar cell) in an enclosure (not shown) that protects against body fluids. The encloser (111) covering the electronic and optical devices is shown in FIG. 1(e).

FIG. 1(c) shows Si (83) to silicon (84 bottom having a cavity or recessed/etched region to provide space for electronic and optical components) seal using gold perimeter fence (85) on Si cover (83) and bottom piece 84. Here, bonding of two Si wafers is shown. In one embodiment of FIG. 1(b), the bottom Si piece 84 also serves as the platform 80 (shown as substrate FIGS. 1a and 1b) as well as hosting sensor electronics (81) and optical devices (e.g. light emitting diode 82 and solar cells 91). These components are protected against body fluids using silicon-on-sapphire cover plate (shown in FIG. 1e where sapphire provides access to external light source powering the solar cells 91). The sapphire or SOS replaces upper Si (83) cover plate. The bonding uses Si—Au eutectic via Au—Si perimeter fence 85 (or frame).

In one embodiment, two silicon pieces, both having a gold frame/fence on the polished surface are bonded. The enclosure contains signal processing electronic chip, LED chip, solar cells, and interconnects. In another embodiment, one silicon piece having a gold frame/fence on the polished surface is bonded to silicon-on-sapphire (SOS) piece having a matching gold fence. A cavity is created by etching either in SOS or Si piece to enclose signal processing electronic chip, LED chip, solar cells, and interconnects. This unit is hermetically sealed and will be body fluid resistant. In addition, the sapphire side will permit light transmissions to power solar cells. Process: The bottom silicon piece was etched in potassium hydroxide (KOH) to create a cavity, as shown below. The Au—Si eutectic bonding ensures hermetic seal. After etching and cleaning, a thin layer of gold was evaporated onto the silicon surface using the lift-off process. More gold was subsequently electroplated onto the evaporated gold. Another embodiment is where rings are made of Si with a hole. Si rings are coated with Au and Pt or Ag/AgCl depending on the electrode requirements.

Our test devices consisted of two silicon pieces, both having a gold frame on the polished surface. The bottom silicon piece was etched in potassium hydroxide (KOH) to create a cavity, as shown. After etching and cleaning, a thin layer of gold was evaporated onto the silicon surface using the lift-off process. More gold was subsequently electroplated onto the evaporated gold.

FIG. 1(d) shows a ring (one of 103, 104, and 105) in FIG. 1b) which is made of material selected from Si, Gold or Pt. The ring can be fitted on the platform (80 also called substrate) of FIG. 1b or 1a. For example, for Si rings one can deposit either Pt or Au or Ag/AgCl (87) or combination depending on if it is configured as working, counter or reference electrode. For example, the Pt working electrode has an interference blocking membrane (polyphenol, OPD, PPD not shown), followed by oxidase enzyme (e.g. GOₓ) cross-linked using glutaraldehyde, polyurethane layer, catalase, followed by drug delivery hydrogel (for reduction of foreign body response). Reference 3 shows an embodiment of various coatings. The reference electrode (103) is Ag/AgCl and the counter electrode is Pt.

FIG. 1(e) shows the sealing using Silicon-on-Sapphire (SOS) cap or cover (111) on the platform (80) of FIG. 1(b). An appropriate region of sapphire in the SOS cap 111 is etched to give place for electronic and optical components (82, 91) mounted on the substrate 80. In concept it is similar to that shown in Si bottom piece 84 in FIG. 1(c).

FIG. 1(f) shows an embodiment where the rings (103, 104, and 105) are placed on top of the SOS cap (111) and substrate 80. This makes the biosensor platform more compact. In one version the SOS cap and bottom substrate are enclosed in a PTFE tubing (not shown).

FIG. 1(g) schematically shows a biosensor platform integrating electrodes protruding as wires that are coated appropriately to serve as reference, working and counter electrodes. This is similar to FIG. 1(e) with the difference that three wires are protruding which serves as three electrodes 103, 104, and 105. They are respectively connected electrically to 100, 101, and 102 electrical connections that goes to electronic and optical components.

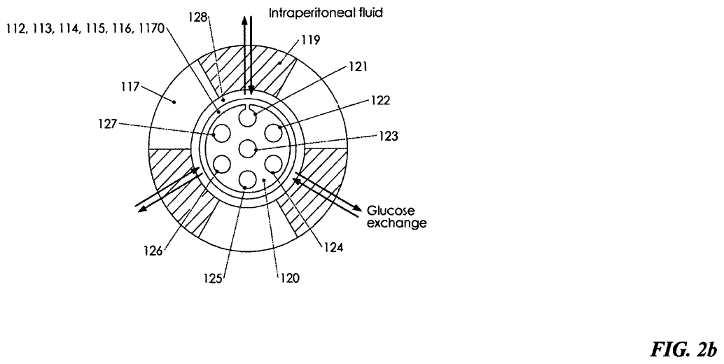

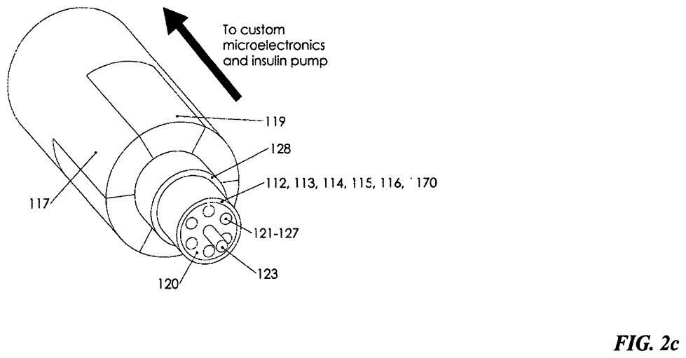

FIG. 2(a) Schematic representation (not to scale) of a six-electrode (112, 113, 114, 115, 116, 1170) glucose sensor for intraperitoneal applications. This six-electrode glucose sensor is outfitted in an insulin catheter (117). In one of the embodiments the overall intraperitoneal (IP) sensor design is based on a train-like configuration where the six cylindrical ring electrodes (112-116, 1170) are lined up within the insulin catheter (117). The electrodes are protected by a stent-like protective cage (shown as dotted lines 118). The insulin delivery tube (118) is coated with a drug coating 119 (e.g. Dexamethasone). The six electrodes have their respective wires forming a bundle 120. RTV (121) is used as a sealant. Another embodiment shows alternate configuration where six ring electrodes (112-116, 1170) are around the insulin delivery tube, 119.

FIG. 2(b). Components from the center outward are insulin dispensing catheter 123 (shown White); Silicone multi-lumen tubing (Blue 120); Platinum six wires (121, 122, 124, 125, 126 and 127 inside silicone tubing 120) and rings (112, 113, 114, 115, 116, 1170) shown gray are wrapped around the silicone tubing 120; Glucose sensing layers (Orange 128); Drug eluting coating (119) embedded on an outer silicone catheter (119 Green/Purple). Glucose exchanges between the IP fluid and the sensing elements (Red arrows).

FIG. 2(c) 3-Dimensional schematic of six electrodes. The sensor components that will be optimized here are the sensing layers (orange 118), drug coating (green 119), outer catheter (purple 117), relative distance between the six electrodes (112, 113, 114, 115, 116, 1170) and electrode size. The inner insulin catheter (119) with the multi-lumen silicone tubing (120) and the platinum rings (112, 113, 114, 115, 116, and 1170) are being developed in parallel in a manufacture-friendly process [Reference 2].

In one embodiment, FIG. 2 can be adapted utilizing heat-shrink fluoro-polymer tubing to connect wire leads to platinum and silver rings (e.g. a six-electrode glucose sensor with multiple working electrodes). We have successfully tested the feasibility of this approach by testing the ability of the six-electrode system with shared counter and reference electrodes to detect hydrogen peroxide, the intermediate for electrochemical glucose sensing.

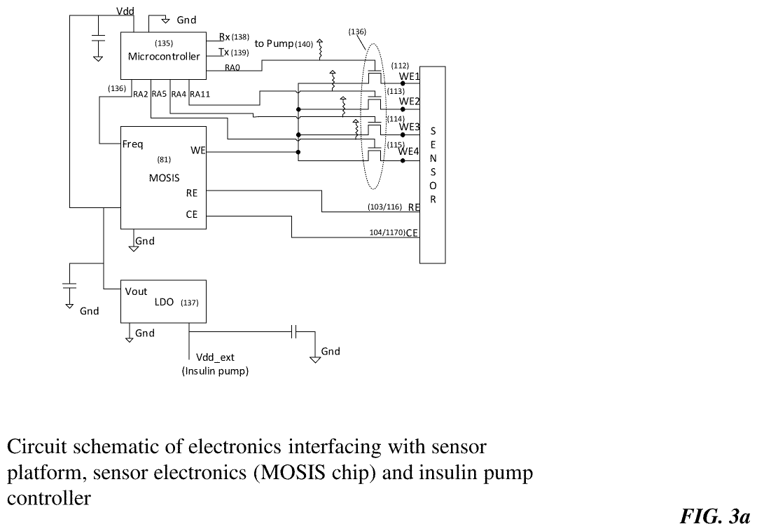

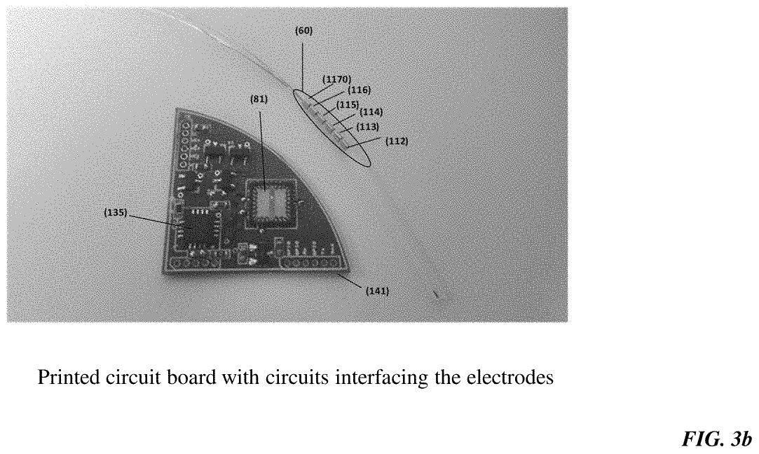

FIG. 3(a) shows is a preliminary circuit schematic used to access sensing electrode. Circuit schematic includes electronics interfacing with sensor platform, sensor electronics (MOSIS chip) and insulin pump controller. Here, four working electrodes (W1 (112), W2 (113), W3 (114) and W4 (115)) and counter electrode CE (1170) and reference electrodes (RE 116) are shown on the platform. These are shown in FIG. 2 as ring electrodes. Microprocessor MCP (P16F688) 135 selects in a sequence four working electrodes by enabling FET switches 136 and electrically connects them to MOSIS chip 81. MOSIS chip block comprises of a Voltage regulator (LDO), a, potentiostat, signal processing unit (SPU), and a buffer. The output signal which is frequency is fed to RA2 pin of the microprocessor MCP 135. MCP 700 has a LDO (137) voltage regulator which interfaces via with Rx (136, 138) and Tx (139) the insulin pump controls (140).

FIG. 3(b) shows the printed circuit board (141). In one version, a printed circuit board that was outfitted with our custom ASIC (81) for SC glucose biosensors and a microchip-based microcontroller (81) for individual control of each sensing element (112-117). This preliminary design provides the basis for the following tasks that will determine the operating parameters of the IP glucose sensor (60).

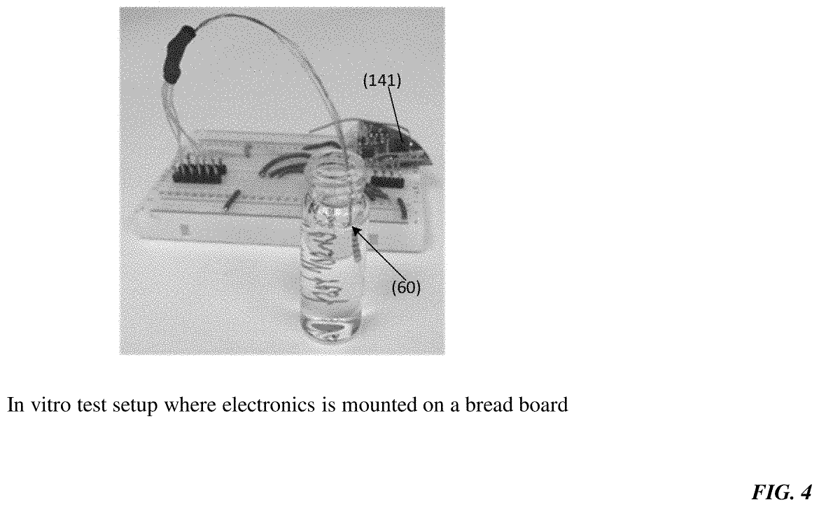

FIG. 4 shows in vitro test set up. Preliminary data: We have prototyped a six-electrode glucose sensor utilizing heat-shrink fluoro-polymer tubing to connect wire leads (121-127) to platinum and silver rings (112, 113, 114, 115, 116, 1170) (FIG. 2). We have successfully tested the feasibility of this approach by testing the ability of the six-electrode system with shared counter and reference electrodes to detect hydrogen peroxide, the intermediate for electrochemical glucose sensing. In this preliminary testing, we designed a printed circuit board that was outfitted with our custom ASIC for SC glucose biosensors (60) and a microchip-based microcontroller (141) for individual control of each sensing element. This preliminary design provides the basis for the following.

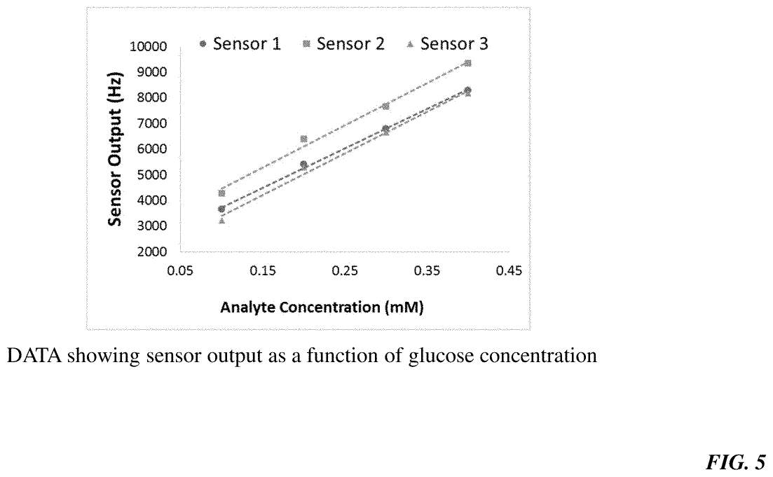

FIG. 5 DATA showing sensor output as a function of glucose concentration.

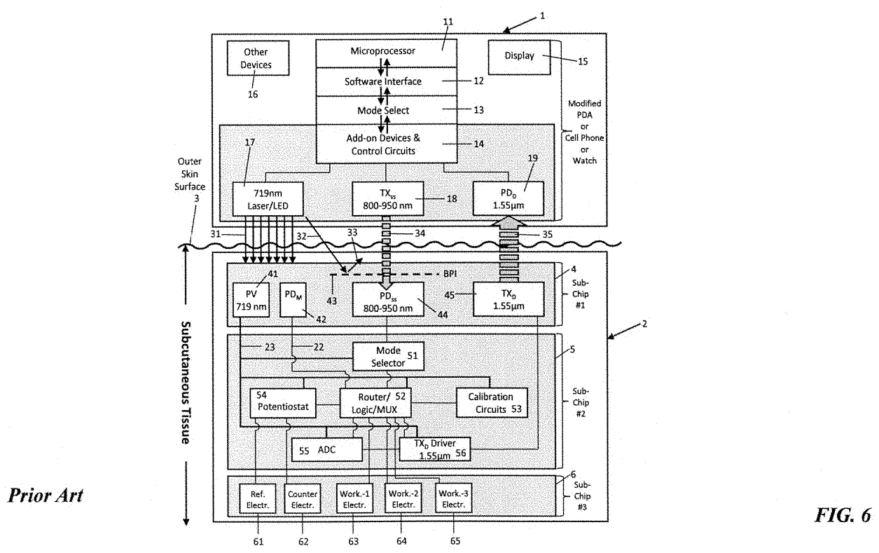

FIG. 6 shows prior art of implantable platform for subcutaneous applications.

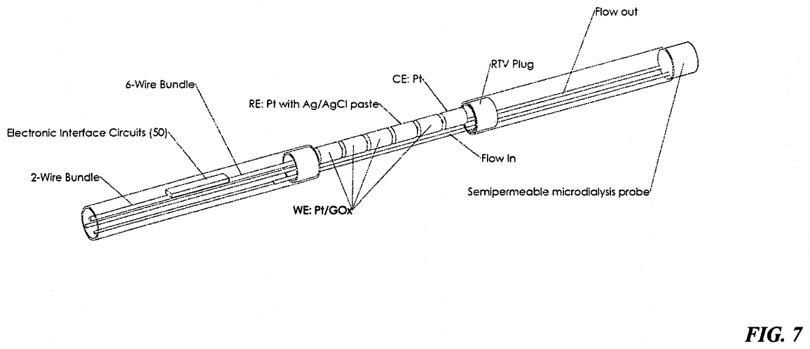

FIG. 7 Overview of a microdialysis catheter setup for subcutaneous bio-sensing. This figure is similar to FIG. 2(a). The difference is that electron interface circuit block 50 is shown embedded in the catheter. Here, six wires coming in results in two wires via the electronic interface circuits.

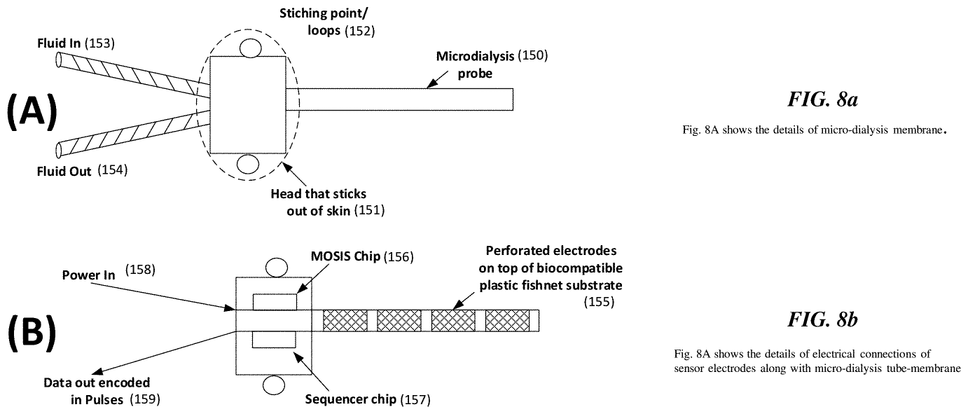

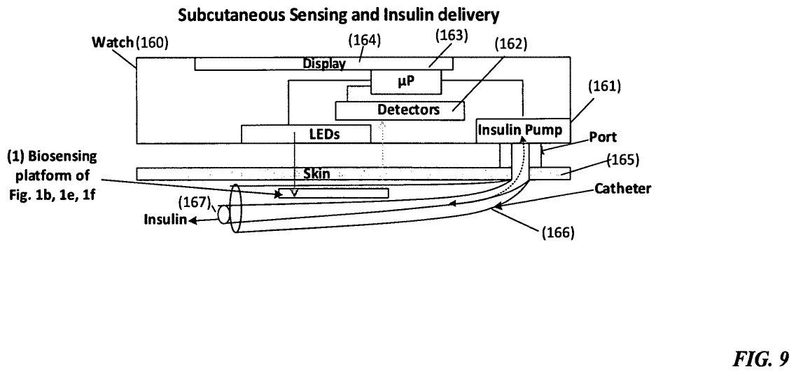

FIG. 8(a) Schematic of microdialysis setup which is housed in a catheter shown in FIG. 7. It shows fluid in 153 and fluid out 154 tubes interfacing with permeable microdialysis probe 150.

FIG. 8(b) Schematic of microdialysis setup housed in a catheter with sensing electrodes in a fish net type housing 155. Power in 158 and data out 159 are schematically shown. Electronic chip 156 and sequencer chip 157 are shown.

FIG. 9. External watch-like proximity communicator unit 160 interfacing with subcutaneous implantable biosensor platform 1 and its electronic and optical devices along with insulin dispensing tube 167 housed in a catheter 166. Here, except for the electronic circuits 50, other components are similar to those in FIG. 2(a).

In accordance with one embodiment of the present invention, a miniaturized, implantable platform, comprising of glucose and other analyte biosensors, sensor electronic and optical interface devices for signal processing, powering and communicating with an external unit in vicinity of the platform is provided. It should be appreciated that the biosensor platform may include at least one electrochemical biosensor that may be exposed to body fluids, as well as one or more sub-components. Accordingly, depending on the application it is contemplated that some components and/or sub-chips and optical devices are employed. Methodologies to house biosensors in catheter are described for subcutaneous as well as intraperitoneal applications. There are various embodiments of the disclosed biosensors with different coatings that are envisioned.

The details of sensor coatings [ref. 3] are not explicitly included in here. However, drug eluting dexamethasone in PLGA spheres loaded in PVA hydrogel forms the outer coating on enclosure housing electronics and optical devices (biosensing platform) as well as sensor electrodes.

It should be appreciated that while the invention has been described with reference to an exemplary embodiment, it will be understood by those skilled in the art that various changes, omissions and/or additions may be made and equivalents may be substituted for elements thereof without departing from the spirit and scope of the invention. In addition, many modifications may be made to adapt a particular situation or material to the teachings of the invention without departing from the scope thereof. Moreover, it is contemplated that elements of one embodiment may be combined with elements of other embodiments as desired. Therefore, it is intended that the invention not be limited to a particular embodiment disclosed herein as the best mode contemplated for carrying out this invention, but that the invention will include all embodiments (individually and/or combined) falling within the scope of the appended claims and/or information. Moreover, unless specifically stated any use of the terms first, second, etc. do not denote any order or importance, but rather the terms first, second, etc. are used to distinguish one element from another.

1. A biosensing platform, wherein the biosensing platform comprises:
a plurality of biosensors, designed to measure levels of analyte selected one from glucose, lactate, pH, oxygen, and
wherein biosensors interfaces with its electronic and optical devices and circuits which are located on the platform, and wherein the platform is placed subcutaneously in a body tissue, and wherein said biosensor electronics and optical devices are housed in an enclosure, and
wherein said enclosure includes a top cover plate and a bottom substrate configured as a hermetically sealed enclosure, and
wherein hermetically sealed enclosure containing all said components of biosensors except the biosensor electrodes, and wherein said biosensing platform is located in a catheter, and wherein the catheter has an insulin delivery tube, and wherein the insulin delivery tube is connected to an insulin pump,
wherein biosensors in the biosensing platform comprise of one or more of working, counter and reference electrodes and wherein electrodes are in the form of rings, and wherein rings ae made of material selected from gold, Pt, Si, carbon nanotubes,
wherein electrodes have coatings designed for a particular analyte level detection, and wherein the electrodes are exposed to body fluids and are electrically connected with potentiostat, signal processing unit and optical transmitter sending optical pulses whose frequency is related to the analyte level, and wherein except for the electrodes all other units interfacing with them are housed in a hermetically, sealed enclosure, and
wherein the hermetically sealed enclosure has at least one surface optically transparent to permit optical source radiation received by the photovoltaic cells, wherein biosensor platform interfaces with an external unit, and wherein external unit comprising of at least one optical source for powering photovoltaic cells located on biosensor platform, one or more microprocessors, and wherein first microprocessor communicating optically with biosensors located in the catheter, and wherein first microprocessor interfaces with optical detectors which receives optical pulses from the optical transmitter located on the biosensor platform, and wherein the optical pulses are processed and displayed as analyte level on a dedicated display, and wherein the analyte level is stored in a nonvolatile memory interfacing the first microprocessor,
wherein an algorithm is executed in the microprocessor based on glucose and other analyte levels and their variations as a function of time, and
wherein analyte levels and their time variations are communicated to a second microprocessor, and wherein second microprocessor interfaces with an insulin pump, and
wherein a second algorithm is executed to dispense insulin dose, and
wherein an interactive feedback control between biosensors and insulin pump is established.

2. The biosensing platform of claim 1, further comprising a plurality of biosensors, designed to measure levels of analyte selected at least one from glucose, lactate, pH, oxygen, and their electronic and optical devices and circuits on a platform located in a catheter which is placed subcutaneously in a body tissue, and
wherein the insulin pump and associated second microprocessor are located in the external unit.

3. The biosensing platform of claim 1, further comprising a plurality of biosensors, designed to measure levels of analyte selected at least one from glucose, lactate, pH, oxygen, and their electronic and optical devices and circuits on a platform located in a catheter which is placed intraperitoneally in the body, and
wherein catheter comprises of an insulin dispensing tube and a miniaturized biosensor platform, and
wherein the biosensor platform comprises of biosensors with working, counter and reference electrodes and
wherein electrodes have coatings designed for a particular analyte level detection, and wherein the electrodes are exposed to body fluids and are electrically connected with potentiostat, signal processing unit and optical transmitter sending optical pulses whose frequency id related to the analyte level, and wherein except for the electrodes all other units interfacing with tem are housed in a hermetically sealed encloser, and wherein the hermetically sealed enclosure has at least one surface optically transparent to permit optical source radiation received by the photovoltaic cells, and wherein biosensors in the biosensing platform comprise of one or more of working, counter and reference electrodes and wherein electrodes are in the form of rings, and wherein rings ae made of material selected from gold, Pt, Si, carbon nanotubes,

4. The biosensing platform of claim 1, further comprising a plurality of biosensors, designed to measure levels of analyte selected at least one from glucose, lactate, pH, oxygen, and their electronic and optical devices and circuits on a platform located in a catheter which is placed subcutaneously in a body tissue, and
wherein catheter comprises of an assembly comprising of microdialysis membrane,
wherein microdialysis assembly comprise of membrane and fluid in and fluid out tubing, and
wherein said assembly comprises of a biosensor platform, and
wherein the biosensing platform comprises of biosensors with working, counter and reference electrodes and
wherein biosensors in the biosensing platform comprise of one or more of working, counter and reference electrodes and wherein electrodes are in the form of rings, and wherein rings ae made of material selected from gold, Pt, Si, carbon nanotubes,
wherein electrodes have coatings designed for a particular analyte level detection, and wherein the electrodes are exposed to body fluids and are electrically connected with potentiostat, signal processing unit and optical transmitter sending optical pulses whose frequency id related to the analyte level, and wherein except for the electrodes all other units interfacing with tem are housed in a hermetically; sealed encloser, and wherein the encloser has at least one surface optically transparent to permit optical source radiation received by the photovoltaic cells,
wherein catheter interfaces with an external unit, and wherein external unit comprising of at least one optical source for powering photovoltaic cells located on biosensor platform housed in the catheter,
one or more microprocessors, and wherein first microprocessor communicating optically with biosensors located in the catheter, and
wherein first microprocessor communicates optically with implanted platform consisting of biosensors located in the catheter, and
wherein first microprocessor interfaces with optical detectors which receives optical pulses from the optical transmitter located on the biosensor platform, and wherein the optical pulses are processed and displayed as analyte level on a dedicated display, and wherein the analyte level is stored in the dedicated memory interfacing the microprocessor,
wherein an algorithm is executed in the microprocessor based on glucose and other analyte levels and their variations as a function of time, and
wherein analyte levels and their time variations are communicated to a second microprocessor, and wherein second microprocessor interfaces with an insulin pump, and
wherein a second algorithm is executed to dispense insulin dose, and
wherein an interactive feedback control between biosensors and insulin pump is established.

5. The biosensing platform of claim 1, further comprising a plurality of biosensors, designed to measure levels of analyte selected at least one from glucose, lactate, pH, oxygen, and their electronic and optical devices and circuits on a platform located in a catheter which is placed subcutaneously in a body tissue, and
wherein biosensors in the biosensing platform comprise of one or more of working, counter and reference electrodes and wherein electrodes are in the form of wires, and wherein wires ae made of material selected from gold, Pt, Pt alloys, Pt and Au coated with carbon nanotubes,
wherein biosensor platform interfaces with an external unit, and wherein external unit comprising of
at least one optical source for powering photovoltaic cells located on biosensor platform,
one or more microprocessors, and wherein first microprocessor communicating optically with biosensors located in the catheter, and
wherein first microprocessor interfaces with optical detectors which receives optical pulses from the optical transmitter located on the biosensor platform, and wherein the optical pulses are processed and displayed as analyte level on a dedicated display, and wherein the analyte level is stored in a nonvolatile memory interfacing the first microprocessor,
wherein an algorithm is executed in the microprocessor based on glucose and other analyte levels and their variations as a function of time, and
wherein analyte levels and their time variations are communicated to a second microprocessor, and wherein second microprocessor interfaces with an insulin pump, and
wherein a second algorithm is executed to dispense insulin dose, and
wherein an interactive feedback control between biosensors and insulin pump is established.

6. A method of integrating a plurality of analyte sensors such as glucose and lactate sensors, their electronic and optical devices and circuits on a platform located in a catheter which is placed subcutaneously in a body tissue,
wherein catheter comprises of an insulin dispensing tube and a miniaturized biosensor platform, and
wherein the biosensor platform comprises of biosensors with working, counter and reference electrodes and
wherein electrodes have coatings designed for a particular analyte level detection, and
wherein the electrodes are exposed to body fluids and are electrically connected with potentiostat, signal processing unit and optical transmitter sending optical pulses whose frequency id related to the analyte level, and wherein except for the electrodes all other units interfacing with tem are housed in a hermetically; sealed encloser, and wherein the encloser has at least one surface optically transparent to permit optical source radiation received by the photovoltaic cells,
wherein biosensors in the biosensing platform comprise of one or more of working, counter and reference electrodes and wherein electrodes are in the form of rings, and wherein rings ae made of material selected from gold, Pt, Si, carbon nanotubes,
wherein biosensor platform in the catheter interfaces with an external unit, and wherein external unit comprising of
at least one optical source for powering photovoltaic cells located on biosensor platform housed in the catheter,
one or more microprocessors, and wherein first microprocessor communicating optically with biosensors located in the catheter, and
an insulin pump and its electronic interface,
wherein second microprocessor communicating electrically with the insulin pump, and dispensing insulin at various intervals of time depending on the glucose and other analyte levels,
wherein first microprocessor interfaces with optical detectors which receives optical pulses from the optical transmitter located on the biosensor platform, and wherein the optical pulses are processed and displayed as analyte level on a dedicated display, and wherein the analyte level is stored in the dedicated memory interfacing the microprocessor,
wherein an algorithm is executed in the microprocessor based on glucose and other analyte levels and its their variations as a function of time, and
wherein analyte levels and their time variations are communicated to a second microprocessor, and wherein second microprocessor interfaces with an insulin pump, and
wherein a second algorithm is executed to dispense insulin dose, and
wherein an interactive feedback control between biosensors and insulin pump is established.

7. A method of integrating a plurality of analyte sensors such as glucose and lactate sensors, their electronic and optical devices and circuits on a platform located in a catheter which is placed intraperitoneally in the body,
wherein catheter comprises of an insulin dispensing tube and a miniaturized biosensor platform, and
wherein the biosensor platform comprises of biosensors with working, counter and reference electrodes and
wherein electrodes have coatings designed for a particular analyte level detection, and wherein the electrodes are exposed to body fluids and are electrically connected with potentiostat, signal processing unit and optical transmitter sending optical pulses whose frequency id related to the analyte level, and wherein except for the electrodes all other units interfacing with tem are housed in a hermetically; sealed encloser, and wherein the encloser has at least one surface optically transparent to permit optical source radiation received by the photovoltaic cells,
wherein catheter interfaces with an external unit, and wherein external unit comprising of at least one optical source for powering photovoltaic cells located on biosensor platform housed in the catheter, insulin pump and its electronic interface, one or more microprocessors, and wherein second microprocessor communicating electrically with the insulin pump, and dispensing insulin at various intervals of time depending on the glucose and other analyte levels,
wherein first microprocessor communicates optically with biosensors on biosensing platform located in the catheter, and
wherein first microprocessor interfaces with optical detectors which receives optical pulses from the optical transmitter located on the biosensing platform, and wherein the optical pulses are processed and displayed as analyte level on a dedicated display, and wherein the analyte level is stored in a nonvolatile memory interfacing the microprocessor,
wherein an algorithm is executed in the microprocessor based on glucose and other analyte levels and their variations as a function of time, and
wherein analyte levels and their time variations are communicated to a second microprocessor, and wherein second microprocessor interfaces with an insulin pump, and
wherein a second algorithm is executed to dispense insulin dose, and
wherein an interactive feedback control between biosensors and insulin pump is established.
